(19) United States
(12) Patent Application Publication
(10) Pub. No.: US 2013/0294673 A1
(43) Pub. Date: Nov. 7, 2013

(54) DATA PROCESSOR AND RADIATION TOMOGRAPHY APPARATUS PROVIDED WITH THE SAME

(71) Applicants: Yoshiyuki Yamakawa, Uji-shi (JP); Masanobu Sato, Kizugawa-shi (JP); Ayako Akazawa, Kyotanabe-shi (JP)

(72) Inventors: Yoshiyuki Yamakawa, Uji-shi (JP); Masanobu Sato, Kizugawa-shi (JP); Ayako Akazawa, Kyotanabe-shi (JP)

(73) Assignee: SHIMADZU CORPORATION, Kyoto-shi, Kyoto (JP)

(21) Appl. No.: 13/980,872

(22) PCT Filed: Jan. 24, 2011
(86) PCT No.: PCT/JP2011/000354

§ 371 Date: Jul. 19, 2013

(51) Int. Cl. A61B 5/00

(52) U.S. Cl. CPC A61B 5/0033
USPC 382/131

(57) ABSTRACT

Provided is a data processor used for imaging a plurality of subjects collectively. In the data processor, trimming is automatically performed to spatial data containing data on three-dimensional information for enhancing working efficiencies of experiments. Specifically, data on the subjects contained in the spatial data is divided into individual divisional data automatically and collectively. This eliminates necessity for individual trimming of the cross sectional images by the experimenter, resulting in significantly facilitating the latter image analysis.

### BACKGROUND

1. Field of the Invention

This invention relates to a data processor used for imaging a plurality of subjects collectively and a radiation tomography apparatus provided with the data processor.

2. Description of the Related Art

A radiation tomography apparatus has been known as one example of an apparatus for imaging a subject as an object to be inspected. The apparatus enables to generate a tomogram of the subject, to which an experimenter refers to recognize information on an internal subject structure.

Now, description will be given of a conventional configuration in such a radiation tomography apparatus. As shown in FIG. 14, the apparatus includes a gantry 51 with an opening. The gantry 51 includes inside thereof a detector ring 62. The detector ring 62 detects radiation generated from radiopharmaceutical administered by injection to the subject. The subject is introduced into the opening of the detector ring 62.

In typical physiological experiments, a plurality of experiments is generally conducted with various experimental conditions. Specifically, in the experiments using small animals, the experiments are often conducted to a plurality of animals while procedures for the experiments change little by little to obtain an experimental result indicating a certain tendency. Consequently, imaging with a radiation tomography apparatus for small animals is typically performed to a plurality of small animals.

Once imaging of a plurality of subjects achieves an enhanced working efficiency of experiments. The conventional apparatus includes a holder that allows accommodation of a plurality of subjects. A tomogram is imaged while the holder is placed inside of the gantry 51. See, for example, Japanese Patent Publications No. 2004-121289, 2005-140560, and 2004-140561.

Since the holder accommodates a plurality of subjects, images of the subjects appear in the tomogram. The experimenter conducts various analyses about cross sectional images of the subjects appearing in the tomogram, and derives experimental results.

The conventional construction, however, has the following problem. Specifically, a problem may occur that one tomogram containing a plurality of cross sectional images leads to difficulty in analytical operations. When experiments are conducted with various conditions of subjects, every cross section image of the subject appears differently in the tomogram. For instance, the cross section images appearing in the tomogram differ from one another in luminance. When luminance is controlled for recognizing the tomogram visibly on a basis of bright cross sectional images appearing in the tomogram, the other cross sectional images appearing darkly become still much darker. This causes deteriorated visibility. On the other hand, luminance is controlled on a basis of dark cross sectional images appearing in the tomogram, the other cross sectional images appearing brightly become still much brighter. This also causes deteriorated visibility.

As noted above, when image processing such as luminance control is required for individual cross sectional images of the subjects, the experimenter has to perform trimming to the individual cross sectional images appearing in the tomogram image to generate an image having a single cross sectional image appearing therein.

Such a situation is not limited to the luminance control. Specifically, a similar trimming operation is also required when intensity in occurrence of radiation from radiopharmaceutical is corrected with use of the weight of the subject. Moreover, when an image in which a distribution of radiopharmaceutical within the subject is projected on a virtual plane is generated, a trimming operation is required. Otherwise, other subjects are obstructive and thus an image that enables to be analyzed cannot be obtained.

This invention has been made regarding the state of the art noted above, and its object is to provide a data processor used for imaging a plurality of subjects collectively and a radiation tomography apparatus provided with the data processor, the data processor and the radiation tomography apparatus enabling to enhance working efficiencies of experiments.

### SUMMARY

This invention is constituted as stated below to achieve the above object. One example of this invention discloses a data processor that processes to three-dimensional spatial data outputted from a radiation tomography apparatus. The data processor includes a dividing device configured to divide the three-dimensional spatial data containing data on a plurality of the subjects to generate divisional data containing data on one of the subjects.

### [Operation and Effect]

The example of this invention enables to provide the data processor that allows enhanced working efficiencies of experiments. Specifically, the data processor in the example of this invention includes the dividing device that divides the spatial data containing the data on the plurality of the subjects to generate the divisional data containing the data on one of the subjects. That is, with the example of this invention, a trimming process is performed to the three-dimensional spatial data automatically. Consequently, the data on the subjects containing in the three-dimensional spatial data is divided into the individual divisional data automatically and collectively. As a result, the experimenter does not have to perform the trimming process to the cross sectional images individually. This significantly facilitates the latter image analysis.

In addition, the data processor above includes an input device inputting commands, and a storing device storing divisional forms of the three-dimensional spatial data. When a command is inputted to into the input device to specify the divisional form of the spatial data, the dividing device reads out the specified divisional form from the storing device to perform division. Such a configuration is more preferable.

### [Operation and Effect]

The above construction describes in detail the data processor of this invention. As in the above construction, when the command to specify the divisional form of the three-dimensional spatial data is inputted to the input device, the dividing device performs division in accordance with the specified divisional form. This enables to provide the data processor with higher general-purpose properties.

In addition, in the above-mentioned data processor, the divisional forms stored in the storing device are associated with a type of a holder for holding the subjects whose data is contained in the three-dimensional spatial data. When a command to specify the type of the holder is inputted into the input device, the dividing device performs division while selecting the divisional form in accordance with the holder.

### [Operation and Effect]

The above construction describes in detail the data processor of this invention. As in the above construction, operations of the dividing device are variable by specifying the type of the holder for holding the subjects, resulting in provision of the data processor with higher operability.

In addition, the above-mentioned data processor includes a holder-shape obtaining device configured to obtain a shape of the holder for holding the subjects in accordance with the three-dimensional spatial data, data on the holder being contained in the three-dimensional spatial data. The dividing device performs division in accordance with the shape of the holder obtained by the holder-shape obtaining device. Such configuration is more preferable.

### [Operation and Effect]

The above construction describes in detail the data processor of this invention. As in the above construction, division is variable by obtaining the shape of the holder from the three-dimensional spatial data and specifying the type of the holder obtained, resulting in provision of the data processor with enhanced convenience operating without specifying and inputting the type of the holder by the experimenter.

Moreover, another example of this invention discloses a radiation tomography apparatus taking cross sectional images of a plurality of subjects. The radiation tomography apparatus includes a radiation source configured to emit radiation; a detecting device configured to detect radiation; a data generating device configured to generate three-dimensional spatial data containing data on a plurality of subjects in accordance with output from the detecting device; and a dividing device configured to divide the three-dimensional spatial data to generate divisional data containing data on one of the subjects.

### [Operation and Effect]

The above construction is application of the data processor of this invention to the radiation tomography apparatus. Specifically, the above data processor is applied to the radiation tomography apparatus that obtains the cross sectional images of the subject through radiation transmittance. This allows provision of the radiation tomography apparatus that enables to perform analysis efficiently even upon performing radiography to a plurality of subjects at one time.

Moreover, another example of this invention discloses a radiation tomography apparatus taking cross sectional images of a plurality of subjects. The radiation tomography apparatus includes a detector ring configured to detect radiation emitted from the subjects; a holder disposed in a hollow portion of the detector ring and configured to accommodate the plurality of subjects; a data generating device configured to generate three-dimensional spatial data containing data on the plurality of subjects in accordance with output from the detector ring; and a dividing device configured to divide the three-dimensional spatial data to generate divisional data containing data on one of the subjects.

### [Operation and Effect]

The above construction is application of the data processor of this invention to the radiation tomography apparatus. Specifically, the above data processor is applied to the radiation tomography apparatus that obtains the cross sectional images of the subjects through determination of radiation emitted from the subjects. This allows provision of the radiation tomography apparatus that enables to perform analysis efficiently eve upon performing radiography to a plurality of subjects at one time.

Moreover, the above radiation tomography apparatus is preferably used for radiography for small animals.

### [Operation and Effect]

The above construction describes in more detail a specific aspect of this invention.

### EFFECTS OF THE INVENTION

The examples of this invention enable to provide the data processor that allows enhanced working efficiencies of experiments. That is, with the examples of this invention, the data on the subjects contained in the three-dimensional spatial data is divided into the individual divisional data automatically and collectively. As a result, the experimenter does not have to perform the trimming process to the cross sectional images individually. This significantly facilitates the latter image analysis.

### BRIEF DESCRIPTION OF THE DRAWINGS

FIG. 1 is a function block diagram of a data processor according to Example 1 of this invention.

FIGS. 2 and 3 are schematic views each for illustration of spatial data according to Example 1 of this invention.

FIG. 4 is a schematic view for illustration of indication by a display according to Example 1 of this invention.

FIG. 5 is a schematic view for illustration of an MIP image according to Example 1 of this invention.

FIG. 6 is a function block diagram of a tomographic X-ray apparatus according to Example 2 of this invention.

FIG. 7 is a plan view of a holder according to Example 2 of this invention.

FIG. 8 is a flow chart for illustration of operations of the tomographic X-ray apparatus according to Example 2 of this invention.

FIG. 9 is a sectional view for illustration of operations of the tomographic X-ray apparatus according to Example 2 of this invention.

FIG. 10 is a function block diagram for illustration of a tomography apparatus according to Example 3 of this invention.

FIGS. 11 to 13 are schematic views each for illustration of a data processor according to one modification of this invention.

FIG. 14 is a sectional view for illustration of a conventional tomography apparatus.

### DESCRIPTION OF REFERENCES

D1 spatial data (three-dimensional spatial data)
D2 divisional data
3 X-ray tube (radiation source)
4 FPD (detecting device)
12 spatial data-generating section (data generating device)
13 dividing section (dividing device)
17 holder-shape obtaining section (holder-shape obtaining device)
26 console (input device)
28 memory (storing device)
32 detector ring

### DETAILED DESCRIPTION

Now, description will be given of each of the examples as the best mode for carrying out this invention.

### Example 1

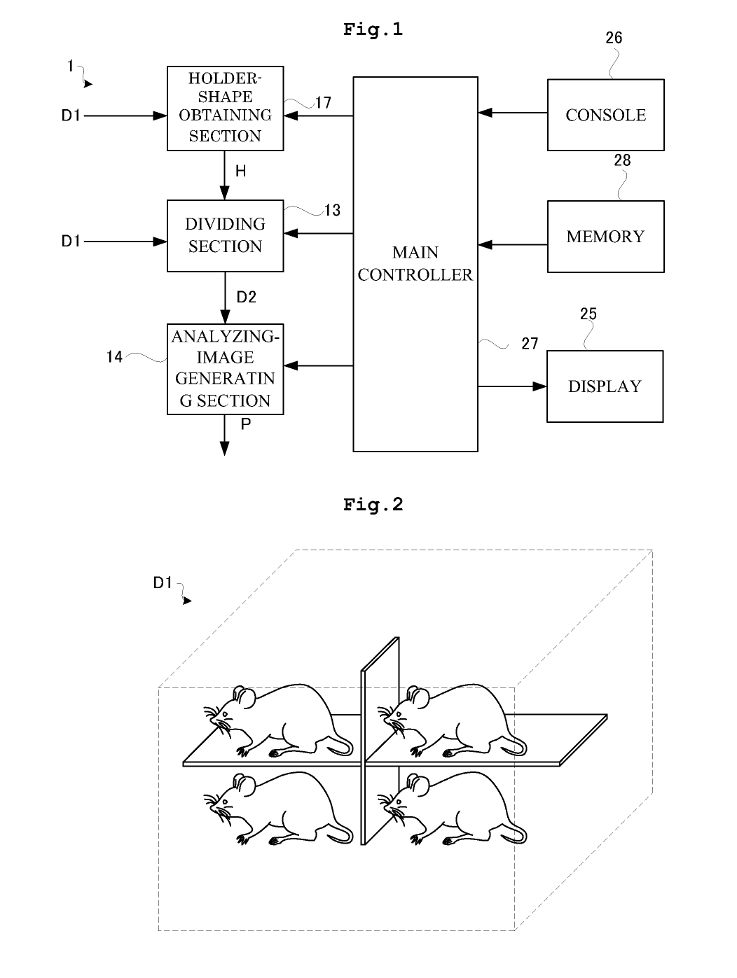

As illustrated in FIG. 1, a data processor 1 according to Example 1 of this invention generates a two-dimensional image P undergoing various types of image processing upon input of spatial data D1 containing data on a plurality of subjects. Here, the spatial data D1 is generated by reconstructing raw data obtained upon imaging a plurality of subjects at one time with use of various types of tomography apparatus. Specifically, the raw data is data such as sinogram or list data. The list data is a data format often used in a PET apparatus to be mentioned later. The spatial data D1 corresponds to the three-dimensional spatial data in this invention.

The data processor 1 according to Example 1 of this invention includes, as illustrated in FIG. 1, a dividing section 13, and an analyzing-image generating section 14. The dividing section 13 divides the spatial data D1 to generate divisional data D2 containing data of a single subject. The analyzing-image generating section 14 generates a two-dimensional image P in accordance with the divisional data D2. The dividing section 13 corresponds to the dividing device in this invention.

As illustrated in FIG. 2, the spatial data D1 is three-dimensional matrix data in which a plurality of subjects (mice) are included in a three-dimensional space. The spatial data D1 is formed of data (e.g. luminance) detected by a radiation tomography apparatus being arranged in each voxel. The spatial data D1 is obtained while a plurality of subjects are introduced into an imaging field of view of the radiation tomography apparatus. The spatial data D1 also indicates a holder holding the subjects. The spatial data D1 includes voxels arranged in a rectangular solid space. The spatial data D1 having a shape of a rectangular solid is advantageous for holding data. The spatial data D1 of a rectangular solid shape includes inside thereof an entire field of view of the radiation tomography apparatus, the field of view having a column shape. Moreover, FIG. 2 illustrates the holder as a divider for dividing the subjects.

As noted above, the spatial data D1 corresponds to the three-dimensional reconstruction data in a step prior to generating a tomogram with the radiation tomography apparatus.

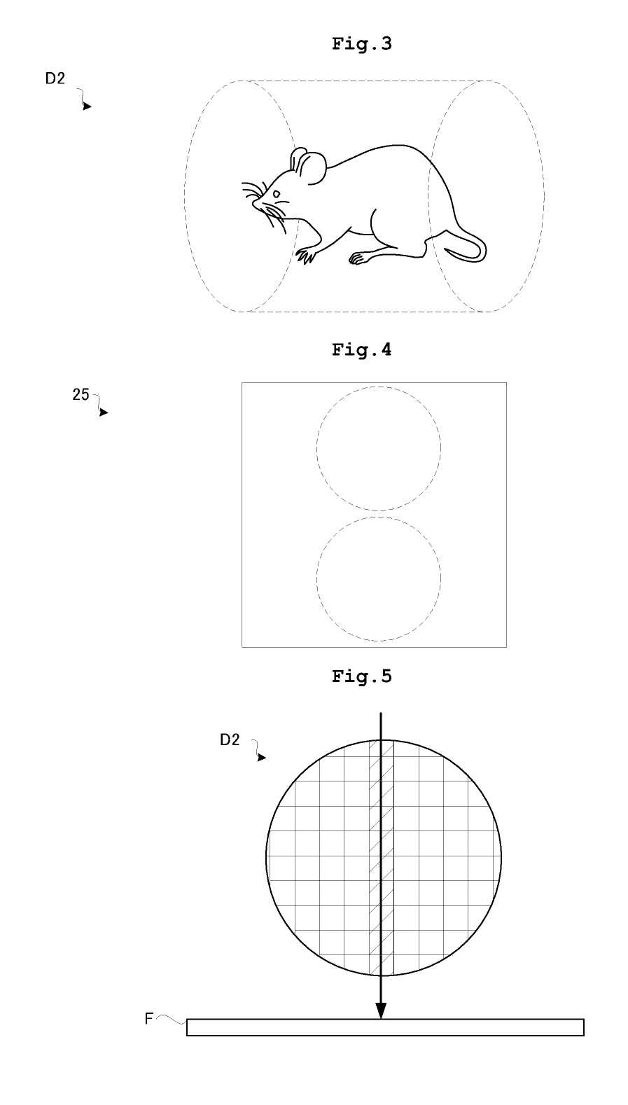

As illustrated in FIG. 3, the divisional data D2 is a three-dimensional matrix data in which a single subject is included in the three-dimensional space. The divisional data D2 is obtained by arranging data detected by the radiation tomography apparatus in each voxel, which is similar to the spatial data D1. The divisional data D2 includes voxels being arranged and the spatial data D1 cut out into a column shape. The voxel of null data may be added outside of the divisional data D2 to shape the divisional data D2 to be rectangular, the divisional data having a column shape for facilitating holding of the data.

The dividing section 13 fetches partially the spatial data D1 to generate the divisional data D2. Such operation causes to convert the spatial data D1 containing data on a plurality of subjects into the divisional data D2 containing a data on one of the subjects. The dividing section 13 generates divisional data D2 for every subject contained in the spatial data D1. As a result, a plurality of pieces of divisional data D2 is generated from the spatial data D1.

A console 26 is provided for input commands by an experimenter (operator). A memory 28 stores the whole information with respect to operation such as parameters to which the dividing section 13 and the analyzing-image generating section 14 refer. The console 26 corresponds to the input device in this invention. The memory 28 corresponds to the storing device in this invention.

Description will be given of operation when the dividing section 13 divides the spatial data D1. The memory 28 stores formats for performing division by the dividing section 13. The memory 28 stores the divisional formats as data indicating coordinates on the spatial data D1 that are fetched as the divisional data D2. Since a plurality of pieces of divisional data D2 are generated from the spatial data D1, the memory 28 is to store the divisional formats for every divisional data D2.

When the experimenter specifies a divisional format through the console 26 in accordance with examination purposes, the dividing section 13 reads out the specified divisional format from the memory 28 to generate a plurality of pieces of divisional data D2 based on the spatial data D1.

Here, at this time, a position of the divisional data D2 may be controlled relative to the spatial data D1. When the experimenter specifies the divisional format through the console 26, a larger rectangle and smaller circles denoted by dotted lines appear on the display 25 displaying cross sectional images as illustrated in FIG. 4. The rectangle indicates the spatial data D1, and the circle inside of the rectangle indicates a position where the divisional data D2 is cut out. The experimenter enables to move the smaller circle appearing on the display 25 through the console 26. When the smaller circle appearing on the display 25 is moved, the dividing section 13 accordingly changes the position of cutting out the divisional data D2 to perform division.

The division by the dividing section 13 for dividing the spatial data D1 is not limited to the above. The dividing section 13 may perform division in accordance with a shape of the holder indicated in the spatial data D1. Specifically, when the experimenter specifies via the console 26 the format (type) of the holder used for radiography with use of the radiography apparatus, the dividing section 13 reads out data stored in the memory 28 with respect to the divisional format from the memory 28 the divisional format being associated with the type of the holder. Thereafter, the dividing section 13 selects the divisional format associated with the type of holder, and performs division in accordance with this selection. Such operation requires adoption of the memory 28 storing the divisional formats in accordance with the type of the holder.

Moreover, the data processor 1 enables to determine the divisional format from the spatial data D1 independently of input by the experimenter. In such the case above, the spatial data D1 is also transmitted to the holder-shape obtaining section 17. See FIG. 1. The holder-shape obtaining section 17 extract the shape of the holder of constructions indicated in the spatial data D1, and transmits coordinate data H to the dividing section 13, the coordinate data H indicating a position of a space in the spatial data D1 where the subject is introduced into the holder. The dividing section 13 performs division in accordance with the coordinate data H. The holder-shape obtaining section 17 determines a shape of the construction indicated in the spatial data D1. When the construction has a plate shape or a column for dividing the space, the holder-shape obtaining section 17 determines the construction as not the subject but the holder. With such the operation, the console 26 and the memory 28 are not always required. The holder-shape obtaining section 17 corresponds to the holder-shape obtaining device in this invention.

The divisional data D2 is transmitted to an analyzing-image generating section 14. The analyzing-image generating section 14 generates a two-dimensional image P with use of the divisional data D2 as three-dimensional matrix data. Examples of the two-dimensional image P to be generated include a tomogram, an SUV image, and an MIP image. Details of these images are to be mentioned later.

The tomogram includes a cross sectional image of the subject appearing therein. The analyzing-image generating section 14 performs data processing, such as luminance adjustment, to the entire divisional data D2, thereby generating a tomogram containing the cross sectional image of the subject when the subject is cut along a certain plane.

The SUV (Standardized Uptake Value) image is a tomogram indicating a distribution of SUV values obtained by normalizing a distribution of radiopharmaceutical. The analyzing-image generating section 14 normalizes the entire divisional data D2 with radioactivity of the radiopharmaceutical administered into the subject and the weight of the subject, thereby obtaining the SUV.

The MIP (Maximum Intensity Projection) image is a two-dimensional image as illustrated in FIG. 5 obtained when the space indicated by the divisional data D2 having a column shape is projected on a plane F. The MIP image is generated as under. Firstly, a straight line is determined that is orthogonal to the plane F at a position on the plane F when an MIP image is to be generated. The maximum value of luminance is selected from each value of luminance indicated by the voxel data (indicated by oblique lines in FIG. 4) through which the straight line passes. Then, the maximum luminance is located in a position on the plane F where the straight line passes. Such operation is also performed to other positions on the plane F, resulting in obtaining an MIP image having the maximum values of luminance for every straight line being arranged in row two-dimensionally. Since the divisional data D2 contains data on only one subject, a plurality of subject is not preferably superimposed upon generating the MIP image.

Moreover, a main controller 27 is provided for performing an overall control of each controller. The main controller 27 has a CPU, and provides each section 13, 14, and 17 by executing various programs.

As noted above, the construction of Example 1 enables to provide the data processor 1 having enhanced working efficiency of experiments. Specifically, the data processor 1 of Example 1 includes the dividing section 13. The dividing section 13 divides the spatial data D1 containing data on a plurality of subjects to generate the divisional data D2 containing data on one of the subjects. That is, with Example 1, trimming process is automatically performed to the spatial data D1. Accordingly, the data on the subject contained into the spatial data D1 is divided into individual divisional data D2 automatically and collectively. This eliminates necessity for individual trimming of the cross sectional images by the experimenter, resulting in significantly facilitating the latter image analysis.

Moreover, as in the above construction, when specification of the divisional format of the spatial data D1 is inputted into the console 26, the dividing section 13 performs division in accordance with the specified divisional format. This achieves provision of the data processor 1 having higher general-purpose properties.

Moreover, as in the above construction, operation of the dividing section 13 varies with specification of the type of the holder for holding the subject. This achieves provision of the data processor 1 with enhanced operability.

Furthermore, as in the above construction, the shape of the holder is obtained from the spatial data D1 and divisional operation varies with specification of the obtained the type of a holder. This achieves provision of data processor 1 with enhanced convenience without inputting the type of a holder by the experimenter.

### Example 2

Next, a radiation tomography apparatus according to Example 2 will be described. The radiation tomography apparatus according to Example 2 is application of the data processor 1 of Example 1 into the CT apparatus. X-rays in Example 2 correspond to the radiation in this invention. An FPD is the abbreviation of a flat panel detector.

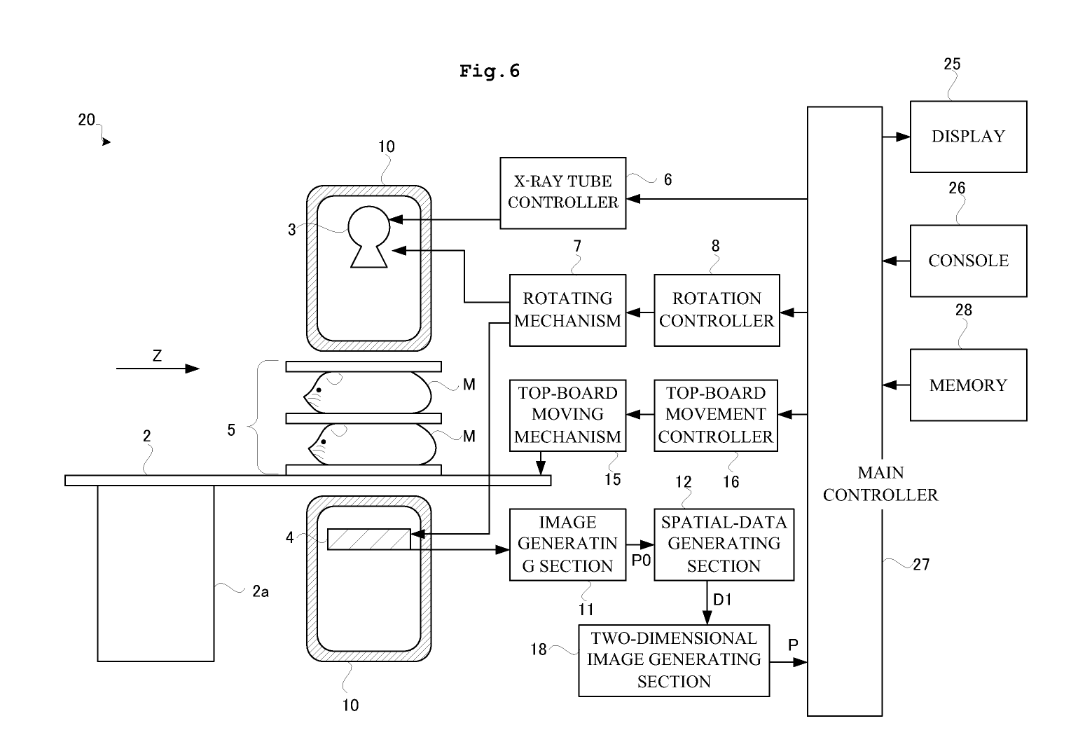

Description will be given first of a tomographic X-ray apparatus according to Example 2. As shown in FIG. 6, a fluoroscopic X-ray apparatus 20 includes a top board 2 for supporting a subject M placed thereon, and a gantry 10 with a through hole extending along the top board 2. The top board 2 is inserted into the through hole of the gantry 10. The top board 2 enables to move forward and backward relative to a support table 2a along the top board 2. The top board 2 is moved by a top-board moving mechanism 15. The top-board moving mechanism 15 is controlled by a top-board movement controller 16.

The gantry 10 includes inside thereof an X-ray tube 3 for emitting X-rays, and an FPD 4 for detecting the X-rays. X-rays from the X-ray tube 3 pass across a through hole of the gantry to reach the FPD 4. Here, the X-ray tube 3 corresponds to the radiation source in this invention. The FPD 4 corresponds to the detecting device in this invention.

An X-ray tube controller 6 is provided for controlling the X-ray tube 3 with a given tube current, a tube voltage, and a pulse width. The FPD 4 detects X-rays emitted from the X-ray tube 3 and transmitting through the subject M, and generates detection signals. The detection signals are sent out to an image generating section 11, where a fluoroscopic image P0 is generated having a projected image of the subject M containing therein. The spatial-data generating section 12 generates the spatial data D1 having luminance values arranged in row three-dimensionally in accordance with the fluoroscopic image P0 generated by the image generating section 11, the luminance value indicating ease of X-ray transmittance. The two-dimensional image generating section 18 is an assembly of the dividing section 13, the analyzing-image generating section 14, and the holder-shape obtaining section 17 of Example 1. Thus, the two-dimensional image generating section 18 is the heart of this invention. When the spatial data D1 is inputted into the two-dimensional image generating section 18, a two-dimensional image P is outputted. The spatial-data generating section 12 corresponds to the data generating device in this invention.

Description will be given of rotation of the X-ray tube 3 and the FPD 4. The X-ray tube 3 and the FPD 4 are rotated integrally by a rotating mechanism 7 about a central axis along the top board 2. A rotation controller 8 controls the rotating mechanism 7.

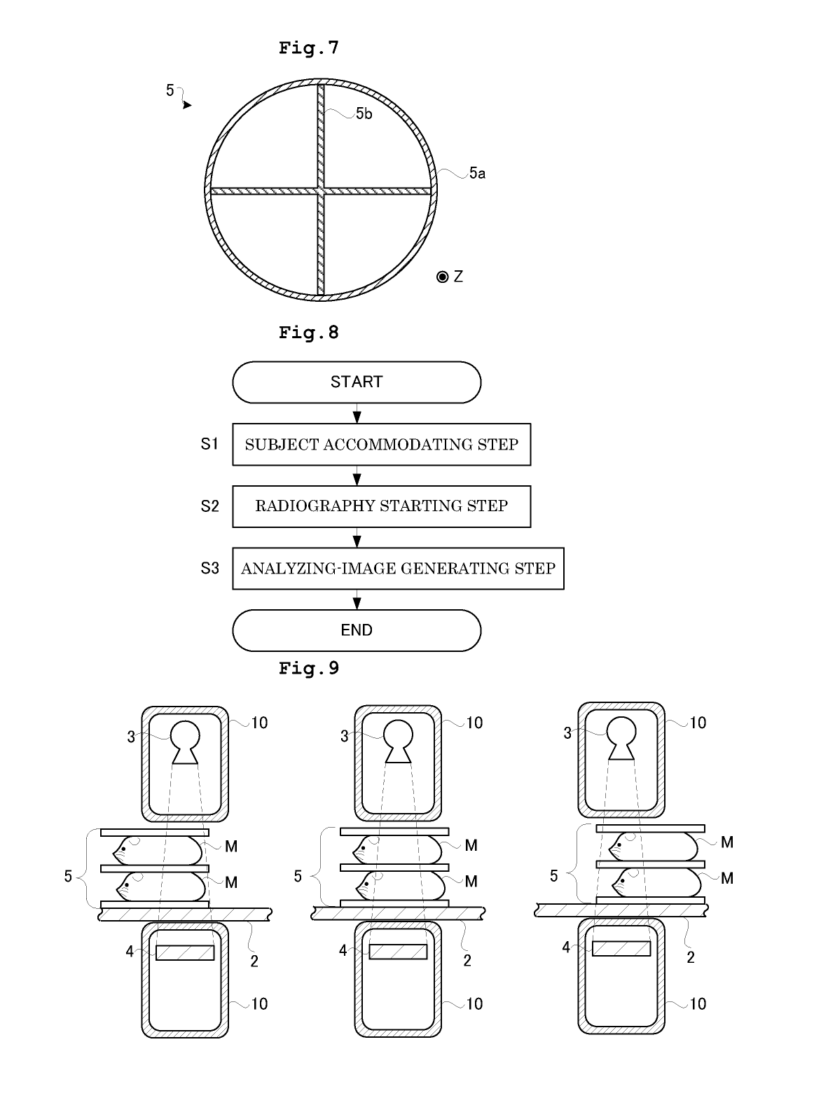

As illustrated in FIG. 7, the holder 5 is cylindrical following the through hole of the gantry 10 having a column shape. Seen the holder 5 in the Z-direction, an outer wall 5a of the holder 5 has a cylindrical shape extending in the-direction. A dividing plate 5b is provided inside the outer wall 5a for dividing an interior of the holder 5a. The dividing plate 5b in FIG. 7, divides the interior of the holder 5 into four parts. The dividing plate 5b extends in the Z-direction. Every one subject M is accommodated in a space divided by the dividing plate 5b such that the subjects M are individually separated by the dividing plate 5b in the-direction. Since the holder 5 extends in the Z-direction, the subjects M may be disposed in series in each of the spaces divided by the dividing plate 5b. Alternatively, the dividing plate 5b may be provided on a plane orthogonal to the Z-direction, the plane dividing every subject M arranged in series. The dividing plate 5b may be varied suitable for radiography purposes or use of the apparatus. The holder 5 is, for example, formed by acrylic resin.

A display unit 25 is provided for displaying the two-dimensional image P obtained through radiography. A console 26 is provided for inputting experimenter's instructions such as start of emitting X-rays. A main controller 27 is also provided for controlling each controller en bloc. The main controller 27 has a CPU, and executes each controller 6, 8, 16 and each section 11, 12, and 18 by executing various programs. The above components may be executed individually respective arithmetic units that perform their functions. A memory 28 stores all parameters with respect to radiography or control of the tomographic X-ray apparatus 20 such as an intermediate image generated with image processing.

### <Operation of Tomography Apparatus>

Next, description will be given of operations of the X-ray tomography apparatus 20. For obtaining a two-dimensional image P for small animals with the tomographic X-ray apparatus 20 according to Example 2, the subject M is accommodated in the holder 5 (subject accommodating step S1), and thereafter radiography of a fluoroscopic image P0 starts (radiography starting step S2), as illustrated in FIG. 8. Thereafter, a two-dimensional image P is generated (analyzing-image generating step S3). Description will be given hereinafter of each of the steps in order.

### <Subject-Accommodating Step S1>

Prior to radiography, a subject M is under anesthesia so as not to move during radiography. A plurality of subjects M is accommodated in the holder 5. Then the holder 5 having the plurality of subjects M accommodated therein is placed on the top board 2.

### <Radiography Starting Step S2>

When the experimenter provides instructions via the console 26 to the tomographic X-ray apparatus 20 to start imaging of a tomogram, the top board 2 slides to introduce the subject M into the through hole of the gantry 10 (see FIG. 6). An X-ray tube controller 6 emits X-rays intermittently in accordance with an irradiation time, a tube current, and a tube voltage stored in the memory 28. Meanwhile, the rotating mechanism 7 rotates the X-ray tube 3 and the FPD 4. The FPD 4 detects X-rays from the X-ray tube 3, the X-rays transmitting through the subject M, and sends detection data at this time to an image generating section 11.

The image generating section 11 images the detection data sent out from the FPD 4, and generates a fluoroscopic imaging P0 having intensity of X-rays being mapped therein. The FPD 4 sends out data to the image generating section 11 in every emission of X-rays from the X-ray tube 3. Accordingly, the image generating section 11 generates a plurality of fluoroscopic images P0. The fluoroscopic images P0 are obtained while the X-ray tube 3 and the FPD 4 move and rotate. Consequently, the fluoroscopic images P0 each contain fluoroscopic images of the subjects M in various perspective directions. The X-ray tube 3 completes emission of X-rays upon one complete rotation of the X-ray tube 3 and the FPD 4 from starting of radiography.

Description will be next given of movement of the top board 2 after starting radiography. The tomographic X-ray apparatus 20 enables to perform radiography to only a portion of the subject M at one time. This is because an imaging field of view of the tomographic X-ray apparatus 20 has a smaller width in the Z-direction than that of the subject M. Accordingly, in Example 2, radiography completed by one rotation of the foregoing X-ray tube 3 and the FPD 4 is performed for a plurality of times, whereby a tomographic image for the entire subject M enables to be obtained. Specifically, as illustrated on left of FIG. 9, radiography is performed to tail sections of the subjects M, and thereafter the top board 2 slides. Accordingly, a relative position between the subject M and the gantry 10 is changed. Then, radiography is performed to abdomen sections of the subjects M. Thereafter, the top board 2 slides again to perform radiography to head sections of the subjects M, as illustrated on the right of FIG. 9. In this way, fluoroscopic images P0 are obtained for the total subject. Here, radiography may be performed firstly to the head section of the subject M.

### <Resolution-Image Generating Step S3>

The fluoroscopic images P0 are sent out to the spatial-data generating section 12. The spatial-data generating section 12 reconstructs a series of fluoroscopic images P0 having information on three-dimensional configurations of the subjects M through imaging in various directions, thereby generating the spatial data D1 in which the subjects M with values of luminance indicating ease of X-ray transmittance being arranged three-dimensionally. The spatial data D1 is transmitted to the two-dimensional image generating section 18, where image processing is performed to every divisional data D2 to generate the two-dimensional image P. Accordingly, the two-dimensional image generating section 18 generates the two-dimensional image P by performing image processing to every subject M independently. The two-dimensional P generated in such way is displayed on the display unit 25, and radiography is completed.

As noted above, the above construction is application of the data processor 1 of Example 1 to the tomographic X-ray apparatus 20. Specifically, application of the above data processor 1 to the tomographic X-ray apparatus 20 in which the cross sectional image of the subject M is obtained by X-ray transmittance achieves the tomographic X-ray apparatus 20 with no decrease of working efficiency of experiments even when the tomographic X-ray apparatus 20 performs radiography to a plurality of subjects M at one time.

### Example 3

Next, description will be given of a radiation tomography apparatus 30 according to Example 3. The radiation tomography apparatus 30 according to Example 3 is incorporation of the data processor of Example 1 into the PET apparatus.

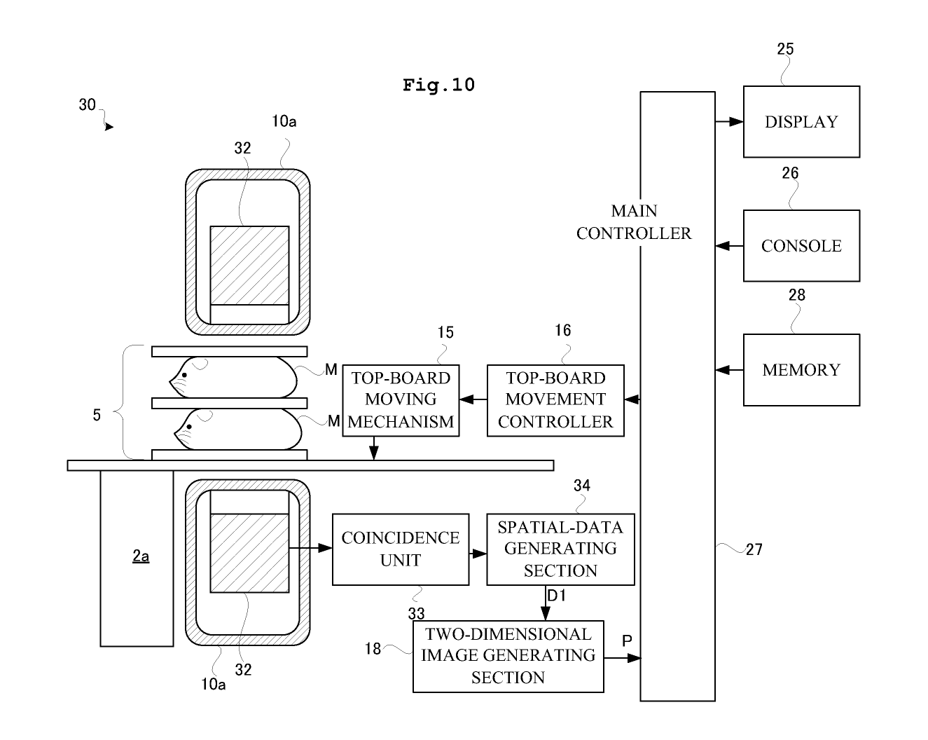

The radiation tomography apparatus 30 includes a gantry 10a as illustrated in FIG. 10. The gantry 10a has a through hole extending in the Z-direction into which the top board 2 is inserted.

The gantry 10a has a hollow along the shape of the gantry 10a, and includes inside thereof a detector ring 32 in a ring shape along the contour of the gantry 10a. The detector ring 32 has detectors arranged in a ring shape that can detect gamma-rays.

A coincidence unit 33 is provided for performing coincidence to detection data outputted from the detector ring 32. Detection frequency and detection positions of annihilation gamma-ray pairs simultaneously entering into a portion in the detector ring 32 can be identified with the coincidence unit 33. The coincidence unit 33 outputs results of coincidence to a spatial-data generating section 34. The spatial-data generating section 34 calculates generating positions of annihilation gamma-ray pairs in accordance with the detection frequency and the detecting position identified with the coincidence unit 33, thereby generating the spatial data D1 having three-dimensionally mapped intensity in occurrence of annihilation gamma-ray pairs. The two-dimensional image generating section 18 is an assembly of the dividing section 13, the analyzing-image generating section 14, and the holder-shape obtaining section 17 of Example 1. Thus, the two-dimensional image generating section 18 is the heart of this invention. Upon input of the spatial data D1 into the two-dimensional image generating section 18, the two-dimensional image P is outputted.

For generation of the two-dimensional image P with use of the radiation tomography apparatus 30, positron emission-type radiopharmaceutical is firstly injected into the subject M. The radiopharmaceutical has a property of concentrating on a specific site, such as a lesion of the subject M. The radiopharmaceutical emits a positron. The positron generates an annihilation gamma-ray pair that travels at a straight angle opposite to each other. Accordingly, an annihilation gamma-ray pair is to be emitted from the subject M. Since a distribution of radiopharmaceutical differs within the subject, the frequency of annihilation gamma-ray pairs differs in sites of the subject M.

A sufficient time lapses from injection of radiopharmaceutical, and then the subject M is anesthetized and housed into the holder 5. Thereafter, the holder 5 having a plurality of subjects M housed therein is placed on the top board 2. When the experimenter provides an instruction via the console 26 to the radiation tomography apparatus 30 to start imaging of a PET image, the top board 2 slides and introduces the subject M into the through hole of the gantry 10a (see FIG. 10). From this time, the detector ring 32 starts detection of the annihilation gamma-ray pair, and the spatial-data generating section 34 generates the spatial data D1 having three-dimensionally mapped intensity in occurrence of annihilation gamma-ray pairs. When the field of view in the z-direction of the radiation tomography apparatus 30 does not entirely cover the total body of the subject M in radiography, the spatial data D1 may be generated while the top board 2 slides in the Z-direction.

The spatial data D1 is transmitted to the two-dimensional image generating section 18, where image processing is performed for every divisional data D2 to generate the two-dimensional image P. Accordingly, the two-dimensional image generating section 18 generates the two-dimensional image P by performing image processing to every subject M. The two-dimensional image generated in such way is displayed on the display unit 25, and radiography is completed.

As noted above, the above construction is application of the data processor 1 of Example 1 to the tomographic X-ray apparatus 30. Specifically, the above data processor 1 is applied to the tomographic X-ray apparatus 30 in which the cross sectional image of the subject M is obtained by determining radiation emitted from the subject M. Consequently, the tomographic X-ray apparatus 20 enables to be provided with no decrease of working efficiency of experiments even when the tomographic X-ray apparatus 30 performs radiography to a plurality of subjects M at one time.

This invention is not limited to the foregoing configurations, but may be modified as follows:

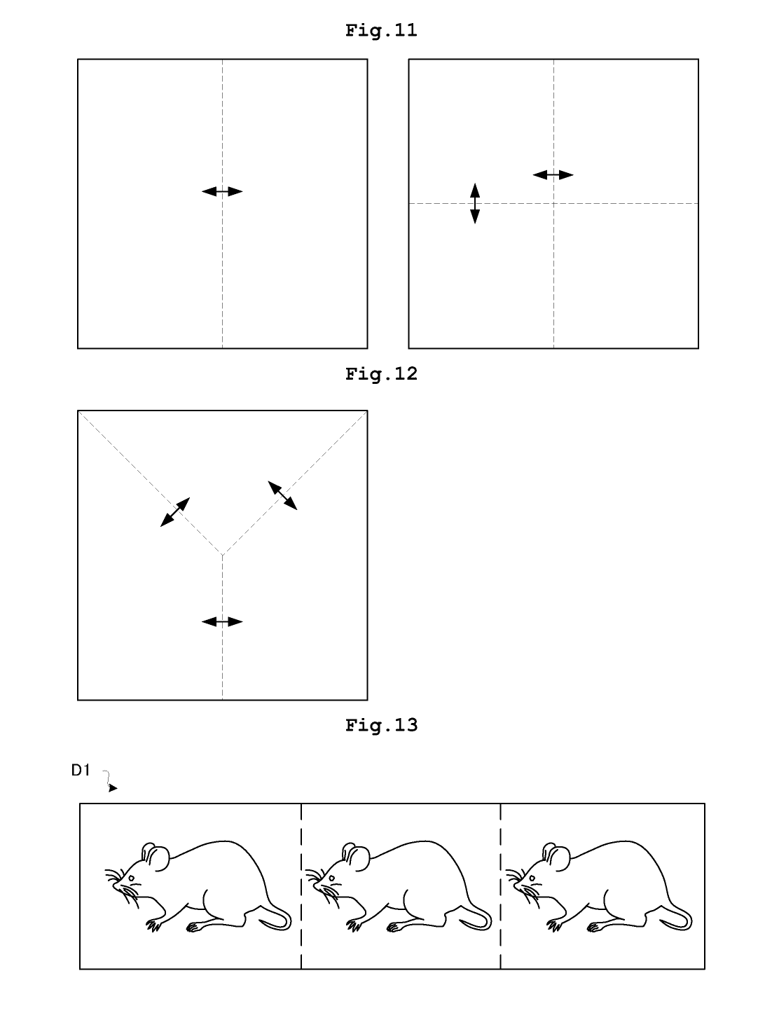

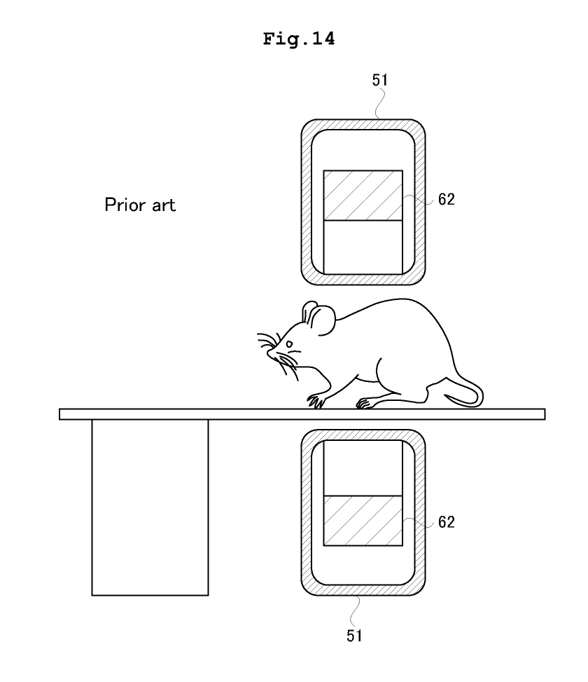

1. With the above construction, the divisional data D2 is generated by cutting out the spatial data D1 into a column shape. This invention, however, is not limited to this. Specifically, the dividing section 13 may generate the divisional data D2 by cutting out the spatial data D1 on a plane instead of operations illustrated in FIG. 4. Here, the display 25 indicates a rectangle expressing the spatial data D1 on the left of FIG. 11 and a straight line expressing a dividing position. The experimenter enables to move the straight line expressing the dividing position through the console 26 as denoted by arrows on the left of FIG. 11. The dividing section 13 recognizes a position specified by the experimenter, and generates the divisional data D2 from the spatial data D1.

2. Moreover, the dividing section 13 may generate the divisional data D2 by cutting out the spatial data D1 on a plurality of planes instead of operations illustrated in FIG. 4. Here, the display 25 indicates a rectangle expressing the spatial data D1 as the left of FIG. 11 and a plurality of straight lines expressing the dividing positions. The experimenter enables to move the plurality of straight lines expressing the dividing positions through the console 26 as denoted by arrows on the right of FIG. 11. The dividing section 13 recognizes a position specified by the experimenter, and generates the divisional data D2 from the spatial data D1.

3. Moreover, the dividing section 13 may generate the divisional data D2 by cutting out the spatial data D1 into a fan shape instead of operations illustrated in FIG. 4. Here, the display 25 indicates a rectangle expressing the spatial data D1 on the left of FIG. 12 and a plurality of straight lines expressing dividing positions. The experimenter enables to rotate the plurality of straight lines expressing the dividing positions through the console 26 as denoted by arrows in FIG. 12. The center of this rotation conforms to a point of intersection of the straight lines indicated on the display 25. The dividing section 13 recognizes a position specified by the experimenter, and generates the divisional data D2 from the spatial data D1.

4. Moreover, the dividing section 13 may generate the divisional data D2 by cutting the spatial data D1 for each piece of data on the subjects M located in series. Specifically, the dividing section 13 divides the spatial data D1 in the position dented by dotted lines in FIG. 13 such that data on the three subjects arranged in series is cut out for every subject as illustrated in FIG. 13, thereby generating the divisional data D2. The experimenter operates the console 26 while viewing the display 25, thereby allowing adjustment of the cutting positions.

5. The data processor according to Example 1 is not limited to application to the X-ray apparatus or a PET apparatus, but may be applied to other tomography apparatus such as an MRI apparatus or a SPECT apparatus.

### INDUSTRIAL UTILITY

As noted above, this invention is suitable for a data processor for researches.

1. A data processor that processes to three-dimensional spatial data outputted from a radiation tomography apparatus, comprising:
a dividing device configured to divide the three-dimensional spatial data containing data on a plurality of the subjects to generate divisional data containing data on one of the subjects.

2. The data processor according to claim 1, comprising:
an input device inputting commands; and
a storing device storing divisional forms of the three-dimensional spatial data, wherein
when a command is inputted to into the input device to specify the divisional form of the spatial data, the dividing device reads out the specified divisional form from the storing device to perform division.

3. The data processor according to claim 2, wherein
the divisional forms stored in the storing device are associated with a type of a holder for holding the subjects, and
when a command to specify the type of the holder is inputted into the input device, the dividing device performs selection of the divisional form in accordance with the holder.

4. The data processor according to claim 1, further comprising:
a holder-shape obtaining device configured to obtain a shape of the holder for holding the subjects in accordance with the three-dimensional spatial data, data on the holder being contained in the three-dimensional spatial data, wherein
the dividing device performs division in accordance with the shape of the holder.

5. A radiation tomography apparatus taking cross sectional images of a plurality of subjects, comprising:
a radiation source configured to emit radiation;
a detecting device configured to detect radiation;
a data generating device configured to generate three-dimensional spatial data containing data on a plurality of subjects in accordance with output from the detecting device; and
a dividing device configured to divide the three-dimensional spatial data to generate divisional data containing data on one of the subjects.

6. A radiation tomography apparatus taking cross sectional images of a plurality of subjects, comprising:
a detector ring configured to detect radiation emitted from the subjects;
a holder disposed in a hollow portion of the detector ring and configured to accommodate the plurality of subjects;
a data generating device configured to generate three-dimensional spatial data containing data on the plurality of subjects in accordance with output from the detector ring; and
a dividing device configured to divide the three-dimensional spatial data to generate divisional data containing data on one of the subjects.

7. The radiation tomography apparatus according to claim 5, wherein
the radiation tomography apparatus is used for radiography for small animals.

8. The radiation tomography apparatus according to claim 6, wherein
the radiation tomography apparatus is used for radiography for small animals.
